SEQUENCE LISTING (24 sequences)

SEQ ID NO: 1 - protein, 6 aa, Artificial
  synthetic peptide
FCIGRL

SEQ ID NO: 2 - protein, 6 aa, Artificial
  synthetic peptide
XCIGRL

SEQ ID NO: 3 - protein, 6 aa, Artificial
  synthetic peptide
FXIGRL

SEQ ID NO: 4 - protein, 6 aa, Artificial
  synthetic peptide
FCXGRL

SEQ ID NO: 5 - protein, 6 aa, Artificial
  synthetic peptide
FCIXRL

SEQ ID NO: 6 - protein, 6 aa, Artificial
  synthetic peptide
FCIGXL

SEQ ID NO: 7 - protein, 6 aa, Artificial
  synthetic peptide
FCIGRX

SEQ ID NO: 8 - protein, 6 aa, Artificial
  synthetic peptide
XXIGRL

SEQ ID NO: 9 - protein, 6 aa, Artificial
  synthetic peptide
XCXGRL

SEQ ID NO: 10 - protein, 6 aa, Artificial
  synthetic peptide
XCIXRL

SEQ ID NO: 11 - protein, 6 aa, Artificial
  synthetic peptide
XCIGXL

SEQ ID NO: 12 - protein, 6 aa, Artificial
  synthetic peptide
XCIGRX

SEQ ID NO: 13 - protein, 6 aa, Artificial
  synthetic peptide
FXXGRL

SEQ ID NO: 14 - protein, 6 aa, Artificial
  synthetic peptide
FXIXRL

SEQ ID NO: 15 - protein, 6 aa, Artificial
  synthetic peptide
FXIGXL

SEQ ID NO: 16 - protein, 6 aa, Artificial
  synthetic peptide
FXIGRX

SEQ ID NO: 17 - protein, 6 aa, Artificial
  synthetic peptide
FCXXRL

SEQ ID NO: 18 - protein, 6 aa, Artificial
  synthetic peptide
FCXGXL

SEQ ID NO: 19 - protein, 6 aa, Artificial
  synthetic peptide
FCXGRX

SEQ ID NO: 20 - protein, 6 aa, Artificial
  synthetic peptide
FCIXXL

SEQ ID NO: 21 - protein, 6 aa, Artificial
  synthetic peptide
FCIXRX

SEQ ID NO: 22 - protein, 6 aa, Artificial
  synthetic peptide
FCIGXX

SEQ ID NO: 23 - protein, 6 aa, Artificial
  synthetic peptide
SLIGRL

SEQ ID NO: 24 - protein, 6 aa, Artificial
  synthetic peptide
SLIGKV

(19) United States
(12) Patent Application Publication
(10) Pub. No.: US 2006/0165722 A1
(43) Pub. Date: Jul. 27, 2006

(54) Peptides for delivery of mucosal vaccines

(75) Inventors: Maria Teresa De Magistris, Rome (IT); Alessio Fasano, West Friendship, MD (US)

Correspondence Address: CONNOLLY BOVE LODGE & HUTZ LLP, SUITE 800, 1990 M STREET NW, WASHINGTON, DC, 20036-3425, US

(73) Assignees: UNIVERSITY OF MARYLAND, BALTIMORE, Baltimore, MD (US); INSTITUTO SUPERIORE DI SANITA, Rome (IT)

(21) Appl. No.: 11/331,278

(22) Filed: Jan. 13, 2006

Related U.S. Application Data
(60) Provisional application No. 60/643,606, filed on Jan. 14, 2005.

(51) Int. Cl. A61K 39/295

(52) U.S. Cl. 424/202.1

(57) ABSTRACT

The present invention is directed to a adjuvant peptide and uses to facilitate antigen absorption in the mucosa, particularly nasal tissue. Vaccine compositions for mucosal delivery include the adjuvant peptide and an antigen for inducing an immune response.

### CROSS REFERENCE TO RELATED APPLICATIONS

This application claims priority to U.S. provisional patent application Ser. No. 60/643,606 filed Jan. 14, 2005, the contents of which are specifically incorporated herein by reference.

### STATEMENT REGARDING FEDERALLY SPONSORED RESEARCH

This invention was made using funds from the United States government, under a grant from the National Institutes of Health DK 048373. The United States government therefore retains certain rights in the invention according to the terms of the grant. This invention was made using funds from the Italian Government, under a grant of the Italian Ministry of Health, “Ricerca Finalizzata” Grant “3AIF” and a grant from the Istituto Superiore di Sanita', Intramural Research Grant “C3MJ.”

### TECHNICAL FIELD OF THE INVENTION

This invention relates to the areas of vaccines and immunotherapy. In particular, the present invention is directed to a nasal dosage composition comprising an adjuvant peptide and an antigen, and methods of using same for mucosal vaccination.

### BACKGROUND OF THE INVENTION

Vaccines have proven to be successful, highly acceptable methods for the prevention of infectious diseases. They are cost effective, and do not induce antibiotic resistance to the target pathogen or affect normal flora present in the host. In many cases, such as when inducing anti-viral immunity, vaccines can prevent a disease for which there are no viable curative or ameliorative treatments available.

As is well known in the art, vaccines function by triggering the immune system to mount a response to an immunogenic agent, or antigen (antigenic agent), typically an infectious organism or a portion thereof that is introduced into the body in a non-infectious or non-pathogenic form. Once the immune system has been “primed” or sensitized to the organism, later exposure of the immune system to this organism as an infectious pathogen results in a rapid and robust immune response that destroys the pathogen before it can multiply and infect enough cells in the host organism to cause disease symptoms. The agent or antigen used to induce the immune system can be the entire organism in a less infectious state, known as an attenuated organism, or in some cases, components of the organism such as carbohydrates, proteins or peptides representing various structural components of the organism.

In many cases, it is necessary to enhance the immune response to the antigens present in a vaccine in order to stimulate the immune system to a sufficient extent to make a vaccine effective, i.e., to confer immunity. Many protein and most peptide and carbohydrate antigens, administered alone, do not elicit a sufficient antibody response to confer immunity. Such antigens need to be presented to the immune system in such a way that they will be recognized as foreign and will elicit an immune response. To this end, adjuvants have been devised which stimulate the immune response.

The best known adjuvant, Freund's complete adjuvant, consists of a mixture of mycobacteria in an oil/water emulsion. Freund's adjuvant works in two ways: first, by enhancing cell and humoral-mediated immunity, and second, by blocking rapid dispersal of the antigen challenge (the “depot effect”). However, due to frequent toxic physiological and immunological reactions to this material, Freund's adjuvant cannot be used in humans. Another molecule that has been shown to have immunostimulatory or adjuvant activity is endotoxin, also known as lipopolysaccharide (LPS). LPS stimulates the immune system by triggering an “innate” immune response—a response that has evolved to enable an organism to recognize endotoxin (and the invading bacteria of which it is a component) without the need for the organism to have been previously exposed. While LPS is too toxic to be a viable adjuvant, molecules that are structurally related to endotoxin, such as monophosphoryl lipid A (“MPL”) are being tested as adjuvants in clinical trials. Currently, however, the only FDA-approved adjuvant for use in humans is aluminum salts (alum) which are used to “depot” antigens by precipitation of the antigens. Alum also stimulates the immune response to antigens.

Thus, there is a recognized need in the art for compounds which can be co-administered with antigens in order to stimulate the immune system to generate a more robust antibody response to the antigen than would be seen if the antigen were injected alone or with alum. Further, because development of mucosal vaccines requires the use of specific adjuvants, adjuvants that work for systemic immunization such as alum are generally not effective for mucosal immunization. Despite intensive research on adjuvants for mucosal vaccines in the last decade, no adjuvants have been registered for human use so far. The main issues in adjuvant research are efficacy and toxicity and candidate mucosal adjuvants do not completely satisfy the criteria of high efficacy and absence of toxicity. Furthermore, most of the proposed mucosal adjuvants are complex molecules whose mechanism of action is poorly understood. Applicants provide herein a non-toxic alternative peptide adjuvant for inducing immune responses to an antigen. The biological activity of this peptide has been well defined and its mechanism of action as an adjuvant has also been studied.

An example of the mucosal adjuvants of the present invention is a peptide of zonula occludens toxin (ZOT; see, for example, U.S. Pat. Nos. 5,665,389; 5,908,825; 5,864,014; 5,912,323; 5,948,629; 5,945,510; and 6,458,925). U.S. Pat. No. 5,908,825 describes a nasal dosage composition for nasal delivery comprising a therapeutic agent and a nasal absorption enhancing effective amount of a purified Vibrio cholera zonula occludens toxin. The purified Vibrio cholera zonula occludens toxin employed is taught to have a molecular weight of about 44 kDa by SDS-PAGE, however, structural information was not known or disclosed. Related U.S. Pat. Nos. 5,864,014 and 5,912,323 further describe the purified Vibrio cholera zonula occludens toxin receptor.

Zonula Occludens Toxin (ZOT) from Vibrio cholerae was identified as an adjuvant for mucosal vaccination (Infect. Immun. 1999, 67:1287; Infect. Immun. 2003, 71:1897). Intranasal administration of ZOT with a soluble antigen in mice stimulated systemic humoral and cell-mediated responses as well as mucosal responses specific for the antigen Ovalbumin (Infect. Immun. 2003, 71:1897). ZOT is a protein of 44.8 kDa that binds a receptor on epithelial cells and modulates tight junctions, inducing the increase of mucosal barrier permeability. The effect of ZOT on tight junctions is reversible and does not cause tissue damage (J. Clin. Invest. 1995, 96:710). The receptor for ZOT on epithelial cells has been partially characterized and recently a mammalian protein with homology to ZOT has been identified and named Zonulin. Interestingly, this protein has been shown to be an endogenous regulator of tight junctions that is released by epithelial cells and binds to the same receptor used by ZOT (Ann. NY. Acad. Sci. 2000, 915:214). The mechanism of ZOT as an adjuvant may involve binding to its receptor on the nasal mucosa, modulation of tight junctions and antigen passage in the submucosa, with subsequent exposure to cells of the immune system.

The development of mucosal vaccines for the prevention of infectious diseases is highly desirable. Mucosal vaccination has several advantages over parenteral vaccination. Mucosal immunization induces an immune response at the site of infection (locally). Furthermore, because of the intrinsic properties of the mucosal immune system, the immunization at one mucosal site can induce specific responses at distant sites (regionally). Such flexibility is important for to address cultural and religious barriers to vaccination because protective immunity (for instance against sexually-transmitted diseases) may then be induced in segregated mucosal sites in a practical way. In addition to local responses against mucosally-acquired pathogens, mucosal vaccines induce systemic immunity, including humoral and cell-mediated responses. Thus, mucosal vaccination could be exploited for combating infections acquired through other routes (i.e., blood or skin). Finally, the administration of mucosal vaccines does not require the use of needles, which could increase vaccine compliance and negate concerns with blood transmissible infections. For all the above reasons mucosal vaccines may be used also to combat cancer, either with preventive or therapeutic vaccination. These vaccines may be both against cancers caused by infectious agents (such as Helicobacter pylori, Papilloma Virus, Herpes Virus) and cancers of different etiology (such as melanoma, colon cancer and others).

Interestingly, most human pathogens are acquired through the mucosal route, however, few mucosal vaccines are presently used. Of those currently used, the vaccine is based on a living attenuated microorganism. Further, purified antigens are not able to stimulate/induce an immune response per se when delivered at mucosal surfaces. Therefore, such vaccines require the use of specific adjuvants. Unfortunately, development of mucosal vaccines has been so far hampered by the lack of safe and effective adjuvants as described above. An effective mucosal adjuvant allows antigen (Ag) passage through a mucosal barrier and facilitates the induction of an Ag-specific immune response.

Applicants disclose adjuvant peptides, e.g., peptides of ZOT, and methods of mucosal delivery of an antigen together with the adjuvant peptide to induce systemic and/or mucosal responses specific for the antigen. Because antigen delivery through the mucosa does not induce an immune response, Applicants determined that co-administration of the ZOT peptide induces systemic and mucosal responses to the antigen. The adjuvant peptide facilitates delivery of the antigen through the mucosa. The adjuvant peptide of the present invention is advantageous in that it is non-toxic, its effects are reversible, it is devoid of endotoxin contamination, readily synthesized and inexpensive to produce and purify.

### SUMMARY OF THE INVENTION

A first embodiment of the invention is a method of inducing an immune response against an antigen in a mammal comprising administering a peptide having amino acid sequence FCIGRL (SEQ ID NO: 1) or a functional derivative thereof and the antigen to the animal, wherein the mammal raises the immune response against the antigen.

A second embodiment of the invention is a method for delivering an antigen through a mucosa of a mammal comprising administering the antigen and a peptide having amino acid sequence FCIGRL or a functional derivative thereof to the mucosa of the mammal.

A third embodiment of the invention is a method for delivering an antigen through a nasal tissue comprising administering the antigen and a peptide having amino acid sequence FCIGRL or a functional derivative thereof to the nasal tissue.

A fourth embodiment of the invention is a method for inducing a systemic response to an antigen comprising administering the antigen and a peptide having amino acid sequence FCIGRL or a functional derivative thereof through the mucosa of a mammal.

A fifth embodiment of the invention is a method for inducing a mucosal response to an antigen comprising administering the antigen and a peptide having amino acid sequence FCIGRL or a functional derivative thereof through the mucosa of a mammal.

A sixth embodiment of the invention is a vaccine composition for inducing an immune response. The vaccine comprises an antigen for inducing an immune response and a peptide having amino acid sequence FCIGRL (SEQ ID NO: 1) or a functional derivative thereof. The vaccine is a mucosal vaccine and delivered to the mucosa of a mammal.

A seventh embodiment of the invention is a method for delivering an antigen to the mucosa of a mammal comprising administering the antigen and a peptide having amino acid sequence FCIGRL (SEQ ID NO: 1) or a functional derivative thereof to the mammal.

In certain embodiments, the administration is intranasally, intravaginally, orally or via intestinal delivery. The administration may be as an aerosol, an inhalant, drops, cream, or the like.

In certain embodiments, the peptide comprises a sequence selected from the group consisting of Xaa₁ Cys Ile Gly Arg Leu (SEQ ID NO: 2), Phe Xaa₂ Ile Gly Arg Leu (SEQ ID NO: 3), Phe Cys Xaa₃ Gly Arg Leu (SEQ ID NO: 4), Phe Cys Ile Xaa₄ Arg Leu (SEQ ID NO: 5), Phe Cys Ile Gly Xaa₅ Leu (SEQ ID NO: 6), and Phe Cys Ile Gly Arg Xaa₆ (SEQ ID NO: 7). The polypeptide is less than 10 amino acid residues in length. Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

In other embodiments, the peptide comprises a sequence selected from the group consisting of: Xaa₁ Xaa₂ Ile Gly Arg Leu (SEQ ID NO: 8), Xaa₁ Cys Xaa₃ Gly Arg Leu (SEQ ID NO: 9), Xaa₁ Cys Ile Xaa₄ Arg Leu (SEQ ID NO: 10), Xaa₁ Cys Ile Gly Xaa₅ Leu (SEQ ID NO: 11), Xaa₁ Cys Ile Gly Arg Xaa₆ (SEQ ID NO: 12), Phe Xaa₂ Xaa₃ Gly Arg Leu (SEQ ID NO: 13), Phe Xaa₂ Ile Xaa₄ Arg Leu (SEQ ID NO: 14), Phe Xaa₂ Ile Gly Xaa₅ Leu (SEQ ID NO: 15), Phe Xaa₂ Ile Gly Arg Xaa₆ (SEQ ID NO: 16), Phe Cys Xaa₃ Xaa₄ Arg Leu (SEQ ID NO: 17), Phe Cys Xaa₃ Gly Xaa₅ Leu (SEQ ID NO: 18), Phe Cys Xaa₃ Gly Arg Xaa₆ (SEQ ID NO: 19), Phe Cys Ile Xaa₄ Xaa₅ Leu (SEQ ID NO: 20), Phe Cys Ile Xaa₄ Arg Xaa₆ (SEQ ID NO: 21), and Phe Cys Ile Gly Xaa₅Xaa₆ (SEQ ID NO: 22). The polypeptide is less than 10 amino acid residues in length. Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

In other embodiments, the peptide adjuvant is SLIGRL (SEQ ID NO:23). In other embodiments, the peptide adjuvant is SLIGKV (SEQ ID NO:24).

In certain embodiments, the present invention is a method of inducing a systemic or a mucosal response to an antigen comprising administering the antigen and a peptide having amino acid sequence selected from the group consisting of SEQ ID NO:23 and SEQ ID NO:24.

In certain embodiments, the present invention is a method of inducing an immune response to an antigen comprising administering the antigen and a peptide having amino acid sequence selected from the group consisting of SEQ ID NO:23 and SEQ ID NO:24.

In one embodiment, the present invention provides methods of inducing an immune response in an animal. Such methods may comprise administering to a mucosa of the animal one or more antigens and one or more peptide adjuvants. In some embodiments, at least one antigen and at least on peptide adjuvant are administered as a composition, for example, antigen and adjuvant may be present in a solution (e.g., an aqueous solution, for example, a saline solution). Compositions may further comprise one or more pharmaceutically acceptable excipients (e.g., salts, buffers, buffer salts, sugars, detergents, talc, and the like). Such methods may be practiced on any type of animal, for example, on a mammal such as a human. Peptide adjuvants for use in the present invention may comprise the sequence FCIGRL and may be from about 6 to about 50 amino acids, from about 6 to about 25 amino acids, or from about 6 to about 15 amino acids in length. Any desired antigen may be used, for example, measles virus antigens, mumps virus antigens, rubella virus antigens, Corynebacterium diphtheriae antigens, Bordetella pertussis antigens, Clostridium tetani antigens, Bacillus anthracis antigens, influenza virus antigens, and combinations thereof. In a particular embodiment, the present invention provides a method of inducing an immune response in an animal (e.g., a mammal such as a human) wherein at least one peptide adjuvant comprises the sequence FCIGRL and the composition is in aqueous solution and the composition comprises one or more antigens selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, and influenza virus antigens.

In another embodiment, the present invention provides immunogenic compositions for mucosal administration. Such compositions may comprise one or more antigens and one or more peptide adjuvants. Such compositions may further comprise one or more pharmaceutically acceptable excipients (e.g., salts, buffers, buffer salts, sugars, detergents, talc, and the like). In some compositions of the invention at least one antigen is selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, and influenza virus antigens. In some compositions of the invention at least one peptide adjuvant comprises the sequence FCIGRL. A peptide adjuvant may be from about 6 to about 50 amino acids, from about 6 to about 25 amino acids, or from about 6 to about 15 amino acids in length. In some embodiments, a composition of the invention may be in aqueous solution (e.g., a saline solution) and may further comprise one or more pharmaceutically acceptable excipients. In a particular embodiment, an immunogenic composition for mucosal administration may comprise at least one peptide adjuvant comprising the sequence FCIGRL and the composition may be in aqueous solution and the composition may comprise at least one antigen selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, and influenza virus antigens.

In another embodiment of the invention, the present invention provides vaccines for mucosal administration. Such vaccines may comprise one or more antigens and one or more peptide adjuvants. Any suitable antigen may be used, for example, antigens selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, influenza virus antigens, and combinations thereof. In some embodiments, a vaccine for mucosal administration may comprise at least one peptide adjuvant comprising the sequence FCIGRL. Suitable peptide adjuvants may be from about 6 to about 50 amino acids, from about 6 to about 25 amino acids, or from about 6 to about 15 amino acids in length. Vaccines for mucosal administration may be in aqueous solution (e.g., saline solution) and may further comprise one or more pharmaceutically acceptable excipients. In a particular embodiment, a vaccine for mucosal administration may comprise at least one peptide adjuvant comprising the sequence FCIGRL and the vaccine may be in aqueous solution and the vaccine may comprise at least one antigen selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, and influenza virus antigens.

In another embodiment, the present invention provides a method of stimulating antigen presenting cells. Such methods may comprise contacting the antigen presenting cells with an adjuvant peptide. Any antigen presenting cell may be stimulated using the methods of the invention, for example, monocytes and/or macrophages may be stimulated. When the antigen presenting cells are human cells, stimulation of antigen presenting cells may result in the antigen presenting cells expressing an increased amount of human major histocompatibility class I and class II molecules and/or CD40. Adjuvant peptides suitable for stimulating antigen presenting cells include, but are not limited to, peptides comprising the sequence FCIGRL. Typically, the adjuvant peptide may be present at a sufficient concentration to stimulate the antigen presenting cells. A sufficient concentration may be from about 0.01 μg/ml to about 500 μg/ml, from about 0.1 μg/ml to about 250 μg/ml, from about 1 μg/ml to about 100 μg/ml, from about 1 μg/ml to about 75 μg/ml, from about 1 μg/ml to about 50 μg/ml, from about 1 μg/ml to about 40 μg/ml, from about 1 μg/ml to about 30 μg/ml, or from about 1 μg/ml to about 20 μg/ml.

These and other embodiments which will be apparent to those of skill in the art upon reading the specification provide the art with reagents and methods for treating and/or preventing diseases.

### BRIEF DESCRIPTION OF THE DRAWINGS

FIG. 1 Dose response curve of the adjuvant AT1002 (AT1002 has the sequence FCIGRL, SEQ ID: 1) after four doses.

FIG. 2 Dose response curve of the adjuvant AT1002 after five doses.

FIG. 3 Comparison of dose response curves of the adjuvant AT1002 after four and five immunizations.

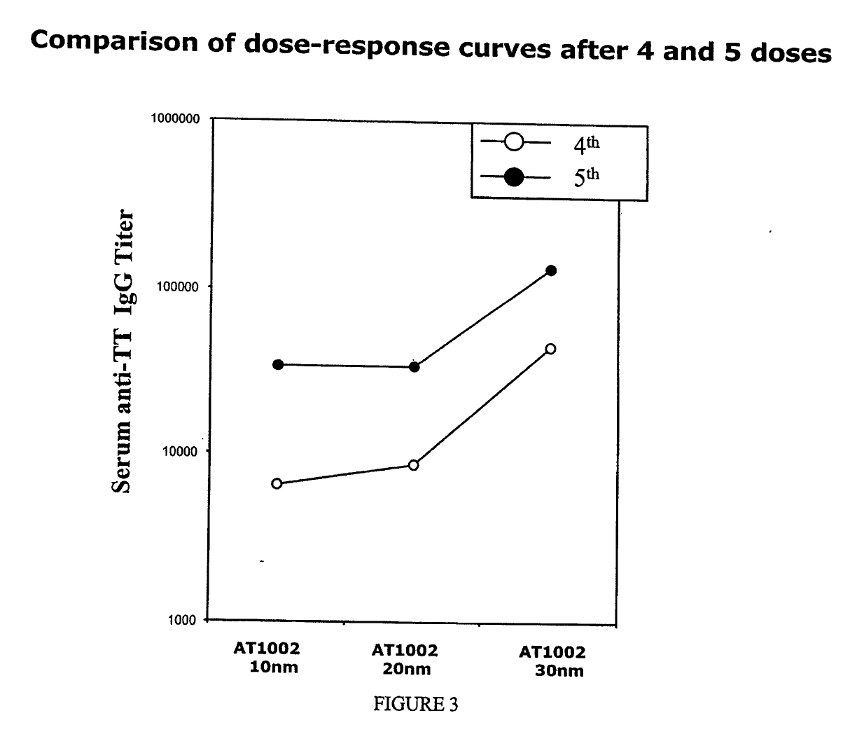

FIG. 4 Serum anti-TT IgA responses induced after six immunization with TT and different doses of the adjuvant AT1002.

FIG. 5 Anti-TT IgA responses induced in vaginal secretions after six immunization with TT and different doses of the adjuvant AT1002.

FIG. 6 is a bar graph showing proliferative responses of splenocytes from mice (C57BL/6) that received four weekly intranasal doses of Tetanus toxoid (TT; 1 μg/dose) alone (white bars) or with TT+AT1002 (22.5 μg/dose, dashed bars) when stimulated with tetanus toxoid.

FIG. 7 shows the results of a FACS analysis of human monocytes stimulated with AT1002 (SEQ ID:1) at the indicated concentrations. After 18 hours the cells were harvested, stained with the indicated monoclonal antibodies and analyzed by FACS.

FIG. 8 shows the results of a FACS analysis of human macrophages stimulated with AT1002 (SEQ ID:1) at the indicated concentrations. After 18 hours the cells were harvested, stained with the indicated monoclonal antibodies and analyzed by FACS.

### DETAILED DESCRIPTION OF THE INVENTION

Defintions

As used herein the specification, “a” or “an” may mean one or more. As used herein in the claim(s), when used in conjunction with the word “comprising”, the words “a” or “an” may mean one or more than one. As used herein “another” may mean at least a second or more.

As used herein, “peptide adjuvant” or “adjuvant peptide” refers to a peptide that functions as an ingredient (as in a composition) that facilitates or modifies the action of the antigen, by inducing, enhancing, and/or boosting the immune response to the antigen.

As used herein, “antigen” refers to any antigenic agent (immunogen) that can elicit an immune response, which can be determined by, for example, production of an antibody that specifically binds to the antigen.

As used herein, “mucosa” refers to a mucous membrane (rich in mucous glands) that lines body passages and cavities which communicate directly or indirectly with the exterior (as the alimentary, respiratory, and genitourinary tracts), that functions in protection, support, nutrient absorption, and secretion of mucus, enzymes, and salts, and that consists of a deep vascular connective-tissue stroma which in many parts of the alimentary canal contains a thin but definite layer of nonstriated muscle and a superficial epithelium which has an underlying basement membrane and varies in kind and thickness but is always soft and smooth and kept lubricated by the secretions of the cells and numerous glands embedded in the membrane. In exemplary embodiments, the mucosa is the mucous membrane of the nose, vagina, rectum, mouth or intestines.

As used herein, “peptide” refers to a peptide of ZOT having amino acid sequence SEQ ID NO: 1 (FCIGRL) and functional derivatives thereof, including but not limited to SEQ ID NOS: 2 through 24. In certain embodiment, the peptide of the present invention is referred to as AT1002 (FCIGRL, SEQ ID: 1).

As used herein, “vaccine” refers to a preparation administered to a subject to produce or artificially increase immunity to a particular disease. The preparation comprises an antigen, such as killed microorganisms, living attenuated organisms, living fully virulent organisms, recombinant biomolecules, immunogenic proteins from a pathogen, antibodies, lipids, polysaccharides, carbohydrates and the like, and a peptide adjuvant.

The Present Invention

Applicants developed a peptide from a Vibrio cholerae phage CTXΦ ZOT protein, which, as disclosed herein, functions as a novel adjuvant peptide. The adjuvant peptide comprises amino acid sequence FCIGRL (SEQ ID NO: 1) and functional derivatives thereof. The adjuvant peptide is less than 10 amino acid residues. The adjuvant peptide may contain only the six amino acids FCIGRL (SEQ ID NO: 1), or it may have additional amino acids. The other amino acids may provide other functions, e.g., antigen tags, for facilitating purification.

Functional derivatives of peptide FCIGRL include, for example, Xaa₁ Cys Ile Gly Arg Leu (SEQ ID NO: 2), Phe Xaa₂ Ile Gly Arg Leu (SEQ ID NO: 3), Phe Cys Xaa₃ Gly Arg Leu (SEQ ID NO: 4), Phe Cys Ile Xaa₄ Arg Leu (SEQ ID NO: 5), Phe Cys Ile Gly Xaa₅ Leu (SEQ ID NO: 6), and Phe Cys Ile Gly Arg Xaa₆ (SEQ ID NO: 7). Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gin; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gin; Xaa₅ is selected from the group consisting of Lys and His; Xaa₆ is selected from the group consisting of Ala, Val, Leu, Ile, Pro, Trp, and Met.

Further, the functional derivative of peptide include: Xaa₁ Xaa₂ Ile Gly Arg Leu (SEQ ID NO: 8), Xaa₁ Cys Xaa₃ Gly Arg Leu (SEQ ID NO: 9), Xaa₁ Cys Ile Xaa₄ Arg Leu (SEQ ID NO: 10), Xaa₁ Cys Ile Gly Xaa₅ Leu (SEQ ID NO: 11), Xaa₁ Cys Ile Gly Arg Xaa₆ (SEQ ID NO: 12), Phe Xaa₂ Xaa₃ Gly Arg Leu (SEQ ID NO: 13), Phe Xaa₂ Ile Xaa₄ Arg Leu (SEQ ID NO: 14), Phe Xaa₂ Ile Gly Xaa₅ Leu (SEQ ID NO: 15), Phe Xaa₂ Ile Gly Arg Xaa₆ (SEQ ID NO: 16), Phe Cys Xaa₃ Xaa₄ Arg Leu (SEQ ID NO: 17), Phe Cys Xaa₃ Gly Xaa₅ Leu (SEQ ID NO: 18), Phe Cys Xaa₃ Gly Arg Xaa₆ (SEQ ID NO: 19), Phe Cys Ile Xaa₄ Xaa₅ Leu (SEQ ID NO: 20), Phe Cys Ile Xaa₄ Arg Xaa₆ (SEQ ID NO: 21), and Phe Cys Ile Gly Xaa₅Xaa₆ (SEQ ID NO: 22). Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gin; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

Any length of peptide adjuvant may be used. Generally, the size of the peptide adjuvant will range from about 6 to about 100, from about 6 to about 90, from about 6 to about 80, from about 6 to about 70, from about 6 to about 60, from about 6 to about 50, from about 6 to about 40, from about 6 to about 30, from about 6 to about 25, from about 6 to about 20, from about 6 to about 15, from about 6 to about 14, from about 6 to about 13, from about 6 to about 12, from about 6 to about 11, from about 6 to about 10, from about 6 to about 9, or from about 6 to about 8 amino acids in length. Peptide adjuvants of the invention may be from about 8 to about 100, from about 8 to about 90, from about 8 to about 80, from about 8 to about 70, from about 8 to about 60, from about 8 to about 50, from about 8 to about 40, from about 8 to about 30, from about 8 to about 25, from about 8 to about 20, from about 8 to about 15, from about 8 to about 14, from about 8 to about 13, from about 8 to about 12, from about 8 to about 11, or from about 8 to about 10 amino acids in length. Peptide adjuvants of the invention may be from about 10 to about 100, from about 10 to about 90, from about 10 to about 80, from about 10 to about 70, from about 10 to about 60, from about 10 to about 50, from about 10 to about 40, from about 10 to about 30, from about 10 to about 25, from about 10 to about 20, from about 10 to about 15, from about 10 to about 14, from about 10 to about 13, or from about 10 to about 12 amino acids in length. Peptide adjuvants of the invention may be from about 12 to about 100, from about 12 to about 90, from about 12 to about 80, from about 12 to about 70, from about 12 to about 60, from about 12 to about 50, from about 12 to about 40, from about 12 to about 30, from about 12 to about 25, from about 12 to about 20, from about 12 to about 15, or from about 12 to about 14 amino acids in length. Peptide adjuvants of the invention may be from about 15 to about 100, from about 15 to about 90, from about 15 to about 80, from about 15 to about 70, from about 15 to about 60, from about 15 to about 50, from about 15 to about 40, from about 15 to about 30, from about 15 to about 25, from about 15 to about 20, from about 15 to about 19, from about 15 to about 18, or from about 15 to about 17 amino acids in length. A peptide adjuvant of the invention may comprise a peptide comprising about 6, about 7, about 8, about 9, about 10, about 11, about 12, about 13, about 14, about 15, about 20, about 30, about 40, about 50, about 60, about 70, about 80, about 90, or about 100 amino acids.

Peptide adjuvants can be chemically synthesized and purified using well-known techniques, such as described in High Performance Liquid Chromatography of Peptides and Proteins: Separation Analysis and Conformation, Eds. Mant et al., C.R.C. Press (1991), and a peptide synthesizer, such as Symphony (Protein Technologies, Inc.); or by using recombinant DNA techniques, i.e., where the nucleotide sequence encoding the peptide is inserted in an appropriate expression vector, e.g., an E. coli or yeast expression vector, expressed in the respective host cell, and purified therefrom using well-known techniques.

The peptide is used to facilitate absorption of an antigen. Further, the absorption occurs through the mucosa, and more particularly through the nasal mucosa. The peptide facilitates absorption across the intestine, the blood-brain barrier, the skin, and the nasal mucosa (See also, copending U.S. application Ser. No. 10/891,492, filed Jul. 15, 2004, published as US 20050059593 herein incorporated by reference in its entirety). Thus the peptide can be formulated with or co-administered with an antigen which targets the nose and/or nasal mucosal tissue. A pharmaceutical composition according to the present invention may be pre-mixed prior to administration, or can be formed in vivo when two agents are administered within 24 hours of each other. Preferably the two agents are administered within 12, 8, 4, 2, or 1 hours of each other.

A “nasal” delivery composition generally comprises water-soluble polymers with a diameter of about 50 μm in order to reduce the mucociliary clearance, and to achieve a reproducible bioavailability of the nasally administered agents. Advantageously, the “nasal” delivery composition is not required to have gastroresistance such as that required for intestinal delivery. The nasal composition comprising polymers are suitable however other excipients are contemplated, provided the peptide adjuvant is permitted to bind to the mucosal membrane.

Nasal dosage compositions for nasal delivery are well-known in the art. Such nasal dosage compositions generally comprise water-soluble polymers that have been used extensively to prepare pharmaceutical dosage forms (Martin et al, In: Physical Chemical Principles of Pharmaceutical Sciences, 3rd Ed., pages 592-638 (1983)) that can serve as carriers for peptides for nasal administration (Davis, In: Delivery Systems for Peptide Drugs, 125:1-21 (1986)). The nasal absorption of peptides embedded in polymer matrices has been shown to enhance through retardation of nasal mucociliary clearance (Illum et al, Int. J. Pharm., 46:261-265 (1988)). Other possible enhancement mechanisms include an increased concentration gradient or decreased diffusion path for peptides absorption (Ting et al, Pharm. Res., 9:1330-1335 (1992)). However, reduction in mucociliary clearance rate has been predicted to be a good approach toward achievement or reproducible bioavailability of nasally administered systemic drugs (Gonda et al, Pharm. Res., 7:69-75 (1990)). Microparticles with a diameter of about 50 μm are expected to deposit in the nasal cavity (Bjork et al, Int. J. Pharm., 62:187-192 (1990)); and Illum et al, Int. J. Pharm., 39:189-199 (1987), while microparticles with a diameter under 10 μm can escape the filtering system of the nose and deposit in the lower airways. Microparticles larger than 200 μm in diameter will not be retained in the nose after nasal administration (Lewis et al, Proc. Int. Symp. Control Rel. Bioact. Mater., 17:280-290 (1990)).

The particular water-soluble polymer employed is not critical to the present invention, and can be selected from any of the well-known water-soluble polymers employed for nasal dosage forms. A typical example of a water-soluble polymer useful for nasal delivery is polyvinyl alcohol (PVA). This material is a swellable hydrophilic polymer whose physical properties depend on the molecular weight, degree of hydrolysis, cross-linking density, and crystallinity (Peppas et al, In: Hydrogels in Medicine and Pharmacy, 3:109-131 (1987)). PVA can be used in the coating of dispersed materials through phase separation, spray-drying, spray-embedding, and spray-densation (Ting et al, supra).

Conventional pharmaceutically acceptable emulsifiers, surfactants, suspending agents, antioxidants, osmotic enhancers, extenders, diluents and preservatives may also be added. Water soluble polymers can also be used as carriers. Other pharmaceutically acceptable carriers and/or diluents are well known in the art to the skilled artisan (see, for example, Remington's Pharmaceutical Sciences, 16th Ed., Eds. Osol, Mack Publishing Co., Chapter 89 (1980); Digenis et al, J. Pharm. Sci., 83:915-921 (1994); Vantini et al, Clinica Terapeutica, 145:445-451 (1993); Yoshitomi et al, Chem. Pharm. Bull., 40:1902-1905 (1992); Thoma et al, Pharmazie, 46:331-336 (1991); Morishita et al, Drug Design and Delivery, 7:309-319 (1991); and Lin et al, Pharmaceutical Res., 8:919-924 (1991)); each of which is incorporated by reference herein in its entirety).

The compositions useful in the methods of the present invention may be administered as an inhalant, liquid drops, aerosols or other formulations that provide for contact of the composition with the mucosa. When administered as a liquid, compositions of the invention may be administered as an aqueous solution, e.g., a saline solution. The parameters of the solution (e.g., pH, osmolarity, viscosity, etc) may be adjusted as necessary to facilitate the delivery of the compositions of the invention. For example, when the aqueous solutions comprise AT1002, it may be desirable to adjust the pH to an acidic pH to enhance the stability of the peptide adjuvant.

The particular antigen employed is not critical to the present invention, and can be, e.g., any biologically active peptide, lipid, polysaccharide, vaccine, or any other moiety otherwise not absorbed through the transcellular pathway, regardless of size or charge.

Examples of vaccines which can be employed in the present invention include peptide antigens and attenuated microorganisms, viruses, parasites and/or fungi. Non-limiting examples of peptide antigens which can be employed in the present invention include the B subunit of the heat-labile enterotoxin of enterotoxigenic E. coli, the B subunit of cholera toxin, diptheria toxin, tetanus toxin, pertussis toxin, capsular antigens of enteric pathogens, fimbriae or pili of enteric pathogens, HIV surface antigens, dust allergens, and acari allergens. Others as are known in the art can also be used, such as, for example, influenza, pertussis, HIV, meningococcal antigens, papilloma virus, bacteria, virus, parasites, fungi and the like. Additional examples of vaccines that can be prepared according to the present invention include, but are not limited to, vaccines comprising antigens (e.g., soluble antigens) derived from cancer, antigens from viruses, bacteria, parasites, fungi, and/or prions. Antigens for use in the vaccines of the invention may be from any source, for example, may be recombinant, synthetic, natural or modified antigens. Antigens may be attenuated or inactivated viruses, bacteria, parasites and/or fungi. Antigens may be recombinant viruses, bacteria, parasites and/or fungi. Antigens may also be recombinant viruses, bacteria, parasites and fungi expressing heterologous vaccine antigens. Antigens may also be allergens.

Examples of attenuated and/or inactivated microorganisms and viruses which can be employed in the present invention include those of enterotoxigenic Escherichia coli, enteropathogenic Escherichia coli, Vibrio cholerae, Shigella flexneri, Salmonella typhi and rotavirus (Fasano et al, In: Le Vaccinazioni in Pediatria, Eds. Vierucci et al, CSH, Milan, pages 109-121 (1991); Guandalini et al, In: Management of Digestive and Liver Disorders in Infants and Children, Elsevior, Eds. Butz et al, Amsterdam, Chapter 25 (1993); Levine et al, Sem. Ped. Infect. Dis., 5:243-250 (1994); and Kaper et al, Clin. Micrbiol. Rev., 8:48-86 (1995), each of which is incorporated by reference herein in its entirety). Examples of cancers include those caused by infectious agents (such as Helicobacter pylori, Papilloma Virus, Herpes Viruses) and cancers of different etiology (such as melanoma, colon cancer, prostate cancer and others).

Any antigen capable of inducing a protective immune response may be used in the vaccines of the invention. Examples of suitable antigens include, but are not limited to, measles virus antigens, mumps virus antigens, rubella virus antigens, Corynebacterium diphtheriae antigens, Bordetella pertussis antigens, Clostridium tetani antigens, Bacillus anthracis antigens, influenza virus antigens, and cancer antigens.

The amount of antigen employed is not critical to the present invention and will vary depending upon the particular ingredient selected, the targeted disease or condition, as well as the age, weight and sex of the subject.

The amount of ZOT peptide employed is also not critical to the present invention and will vary depending upon the age, weight and sex of the subject. Generally, the final concentration of peptide employed in the present invention to enhance absorption of the biologically active ingredient by the mucosa is in the range of about 10⁻⁵ M to 10⁻¹⁰ M, preferably about 10⁻⁶M to 5.0×10⁻⁵ M. By way of example, to achieve such a final concentration, the amount of peptide in a single oral dosage composition, such as for administration to the intestinal mucosa, will generally be about 4.0 ng to 2.5 micrograms, or 4.0 ng to 1000 ng, preferably about 40 ng to 80 ng. In certain embodiments, for example in a mammal of about 20 g, the amount administered of antigen is about 2.5 micrograms and the amount of adjuvant peptide is about 22.5 micrograms (1:10 ratio). In other embodiments, for example in a mammal of about 20 g, the amount administered of antigen is about 2.5 micrograms and the amount of peptide is about 22.5, or about 15, or about 7.5 micrograms.

The ratio of antigen to peptide employed is not critical to the present invention and will vary depending upon the amount of biologically active ingredient to be delivered within the selected period of time and, further, upon the type of mucosae targeted. Generally, the weight ratio of therapeutic or immunogenic agent to peptide employed in the present invention is in the range of about 1:100 to 3:1, or about 1:10 to 2:1. Applicants contemplate that higher amounts of adjuvant peptide relative to antigen induces a relatively stronger immune response systemically and/or in the mucosa targeted.

Conservative substitutions, in which an amino acid is exchanged for another having similar properties, can be made in the peptide having the sequence of SEQ ID NO: 1. Examples of conservative substitutions include, but are not limited to, Gly←→Ala, Val←→Ile←→Leu, Asp←→Glu, Lys←→Arg, Asn←→Gln, and Phe←→Trp←→Tyr. Conservative amino acid substitutions typically fall in the range of about 1 to 2 amino acid residues. Guidance in determining which amino acid residues can be substituted without abolishing biological or immunological activity can be found using computer programs well known in the art, such as DNASTAR software, or in Dayhoff et al. (1978) in Atlas of Protein Sequence and Structure (Natl. Biomed. Res. Found., Washington, D.C.).

Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Particularly preferred peptide analogs include substitutions that are conservative in nature, ie., those substitutions that take place within a family of amino acids that are related in their side chains. Specifically, amino acids are generally divided into families: (1) acidic—aspartate and glutamate; (2) basic—lysine, arginine, histidine; (3) non-polar—alanine, valine, leucine, isoleucine, proline, phenylalanine, methionine, tryptophan; (4) uncharged polar—glycine, asparagine, glutamine, cysteine, serine threonine, and tyrosine; and (5) aromatic amino acids—phenylalanine, tryptophan, and tyrosine. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity.

Any assay known in the art can be used to determine the inventive peptide biological activity. For example, the assay may involve (1) assaying for a decrease of tissue resistance (Rt) of ileum mounted in Ussing chambers as described by Fasano et al, Proc. Natl. Acad. Sci., USA, 8:5242-5246 (1991); (2) assaying for a decrease of tissue resistance (Rt) of intestinal epithelia cell monolayers in Ussing chambers as described below; or (3) assaying for intestinal or nasal enhancement of absorption of a therapeutic or immunogenic agent, as described in WO 96/37196; U.S. patent application Ser. No. 08/443,864, filed May 24, 1995; U.S. patent application Ser. No. 08/598,852, filed Feb. 9, 1996; and U.S. patent application Ser. No. 08/781,057, filed Jan. 9, 1997.

The peptide of the present invention rapidly opens tight junctions in a reversible and reproducible manner, and thus can be used as a nasal absorption enhancer of an antigen, in the same manner ZOT is used (see WO 96/37196; U.S. patent application Ser. No. 08/443,864, filed May 24, 1995; U.S. patent application Ser. No. 08/598,852, filed Feb. 9, 1996; and U.S. patent application Ser. No. 08/781,057, filed Jan. 9, 1997).

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

The following examples demonstrate that mucosal immunization by administering an antigen and a mucosal adjuvant of SEQ ID NO:1 induces serum IgG, induces mucosal IgA in different mucosal districts, and is highly effective as compared to other mucosal adjuvants. Accordingly, AT1002 functions as a mucosal adjuvant and induces an immune response to the antigen in a subject.

### EXAMPLE 1

Intranasal Immunization with Tetanus Toxoid (TT) and ZOT Peptide (AT1002)

Groups of four C57BL/6 female mice were intranasally immunized with Tetanus Toxoid (TT) 2.5 μg alone or with TT plus AT1002 at the dose indicated or with TT plus the known adjuvant heat-labile enterotoxin (LT) as a control.

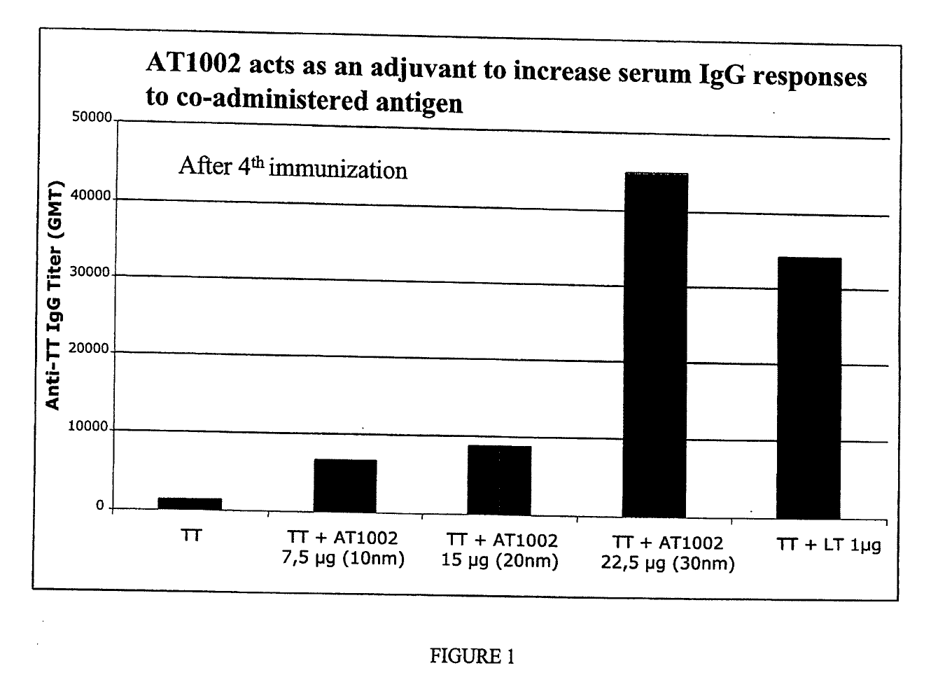

FIG. 1 shows the geometric mean titers of anti-TT serum IgG after four immunizations. The results show that AT1002 acts as an adjuvant in that it elicits serum responses to TT higher as compared to those of animals immunized with TT alone. Furthermore, the results show that the AT1002 dose of 30 nanomoles is relatively most effective.

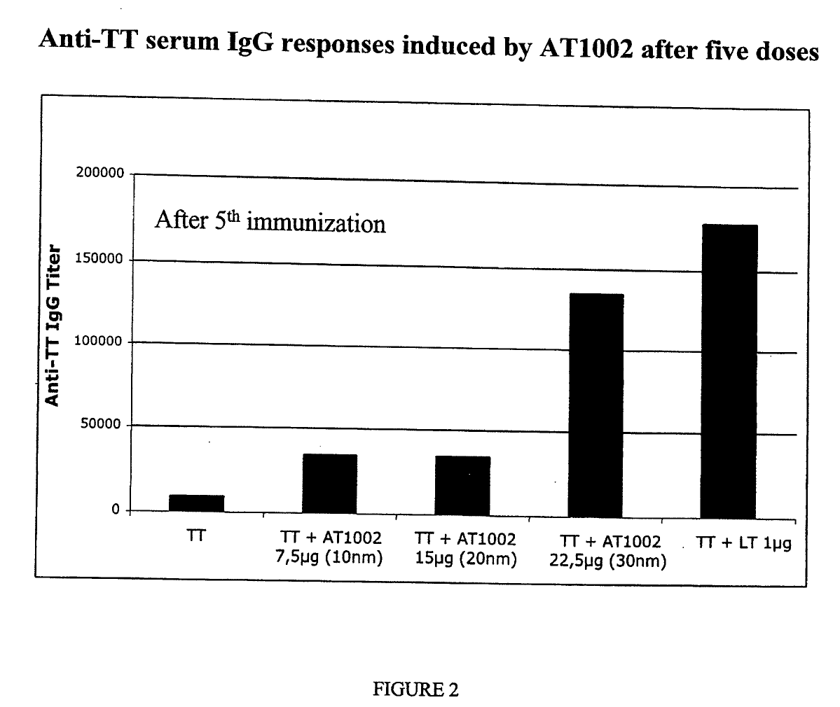

FIG. 2 shows the geometric mean titers of anti-TT serum IgG after four immunizations. These results show that the anti-TT serum responses elicited by AT1002 are higher than those observed after four doses. Again the AT1002 dose of 30 nanomoles is the relatively most effective.

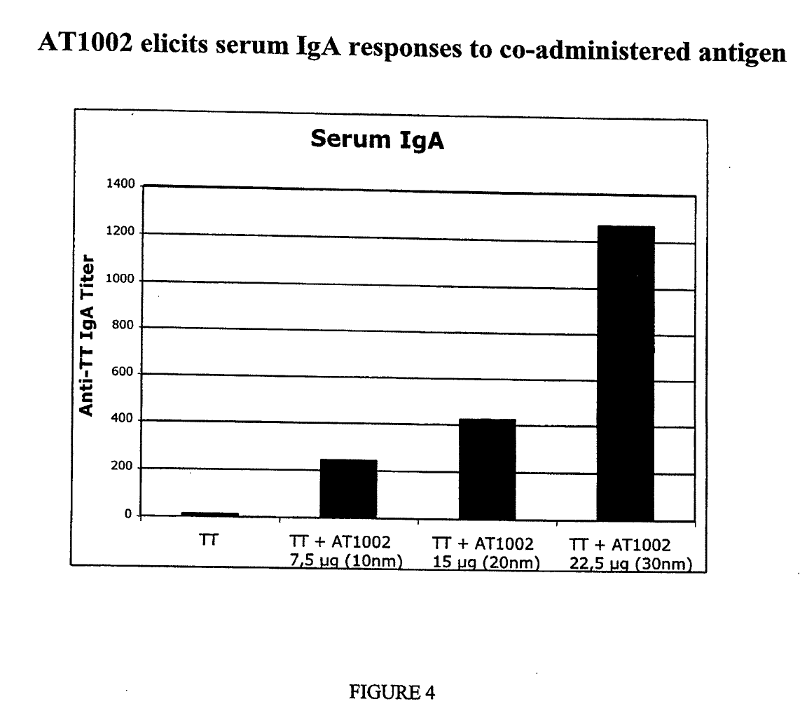

Serum anti-TT IgA responses were determined to be induced after six immunizations with TT and different doses of the adjuvant AT1002 (FIG. 4). Groups of four C57BL/6 female mice were intranasally immunized with Tetanus Toxoid (TT) 2.5 μg alone or with TT plus AT1002 at the dose indicated. The results show the geometric mean titers of anti-TT serum IgA. The data show that AT1002 induces serum IgA against the co-administered antigen. Applicants further contemplate, based on observations, the induced response may occur after one, two, three, four or five immunizations.

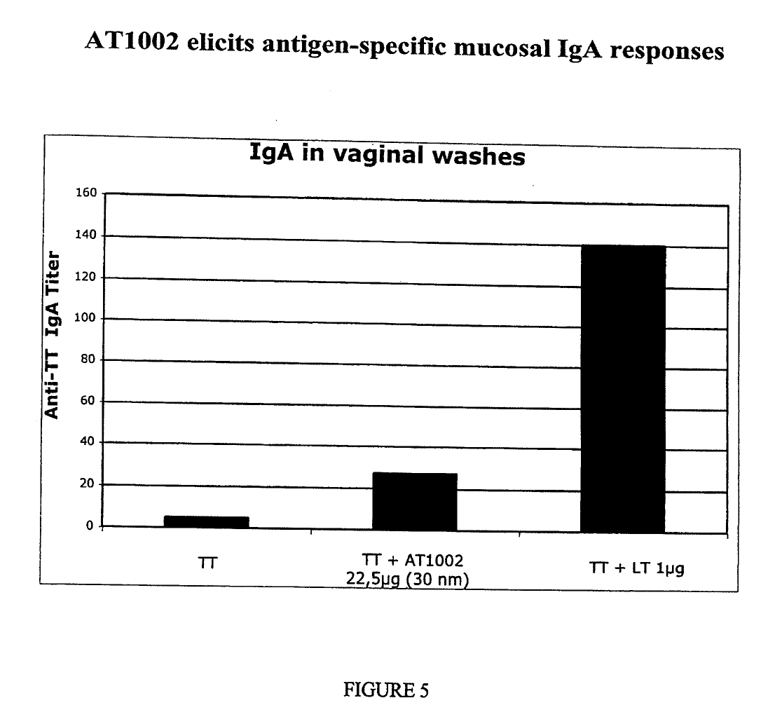

Applicants also observed anti-TT IgA responses were induced in vaginal secretions after six immunizations with TT and different doses of the adjuvant AT1002 (FIG. 5). The results show the geometric mean titers of anti-TT IgA and indicate that AT1002 induces IgA against the co-administered antigen in a mucosal district far from the site of immunization. Applicants further contemplate, based on observations, the induced response may occur after one, two, three, four or five immunizations.

Commercial peptides SLIGRL (mouse, SEQ ID NO:23) and SLIGKV (human, SEQ ID NO: 24) (both commercially available from Sigma) may be employed in the manner described above for AT1002. Briefly, an adjuvant peptide of one of SEQ ID NOS: 23 or 24 may be administered along with an antigen, such as, for example, TT. The number of immunization may be one, two, three, four, five or six. Immune response may be determined, specifically if TT is used, anti-TT IgA and anti-TT IgG titers may be measured in either of the serum and/or vaginal secretions.

### EXAMPLE 2

ZOT Peptide as a Mucosal Adjuvant

The results presented herein demonstrate peptide AT1002 acts as a mucosal adjuvant. More specifically, upon mucosal immunization of a mammal, the co-administration of AT1002 induces serum IgG, IgA in the serum and mucosal IgA in vaginal secretions.

### EXAMPLE 3

AT1002 Induces Protective Responses to the Co-Delivered Antigen.

Mice (C57BL/6) received four weekly intranasal doses of Tetanus toxoid (TT; 1 μg/dose) with or without AT1002 (30 μg/dose) and 2 months later the mice were challenged subcutaneously with DP50 (50 times the dose paralyzing 50% of the animals, as established in preliminary experiments) of tetanus toxin and paralysis and death were monitored for one week. The results in Table 1 show that the mice immunized with TT alone were not protected whereas the mice that received the antigen with AT1002 were all protected. Furthermore, the serum IgG titers specific for the antigen were analyzed in individual mice immediately before the challenge. The range of the titers measured is reported in the Table.

TABLE 1
Survival of intranasally immunized mice to Tetanus Toxin challenge

| Vaccine | No. of survivors/No. of mice | range of anti-TT IgG titer |
|---|---|---|
| TT alone | 0/7 | 256-4,096 |
| TT + AT1002 | 8/8 | 16,384-65,536 |

These results demonstrate that: a) AT1002 induces protective responses to the co-administered antigen; b) mucosal (intranasal) immunization with AT1002 induces protective responses against a systemic (subcutaneous) challenge; and c) AT1002 induces “memory” protective responses as the challenge was performed two months after the last vaccination dose. Indeed, the anti-TT serum IgG titers after two months were high. (Note that two months is a significant amount of time for the mouse lifespan).

### EXAMPLE 4

AT1002 Induces Cell-Mediated Responses

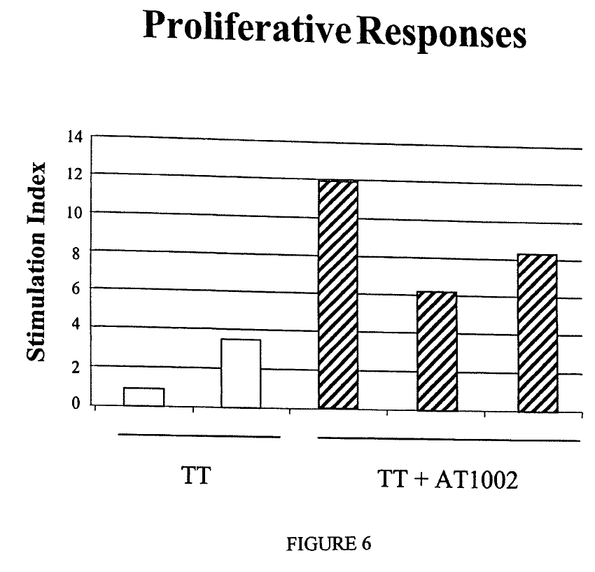

With reference to FIG. 6, mice (C57BL/6) received four weekly intranasal doses of Tetanus toxoid (TT; 1 μg/dose) alone (white bars) or with TT+AT1002 (22.5 μg/dose, dashed bars). Spleens were removed one week after the last dose and splenocytes were tested in proliferation assays where TT was added to cultures and tritiated thymidine incorporation was measured. The Stimulation Index (cpm of cultures with TT/cpm of cultures without TT) values show that the mice immunized with TT+AT1002 proliferated to the antigen whereas the mice immunized with TT alone did not (values equal or above four were considered positive).

These results demonstrate that AT1002 induces cell-mediated responses against the co-administered antigen. Thus, antigen-specific T lymphocytes are primed by mucosal immunization with AT1002 as an adjuvant.

### EXAMPLE 5

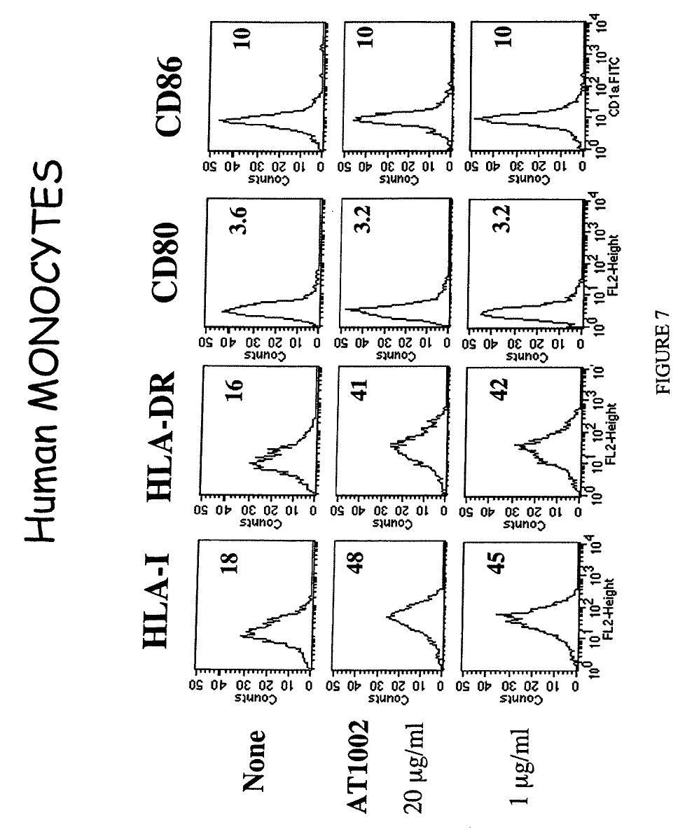

Human monocytes were purified from peripheral blood of healthy donors and cultured in complete medium. After 2 hours the stimuli were added to cultures and after 18 hours the cells were harvested, stained with the indicated monoclonal antibodies and analyzed by FACS. The results are shown in FIG. 7.

FIG. 7 demonstrates that AT1002 has an immunopotentiating effect on human antigen presenting cells such as monocytes and macrophages. FIG. 7 shows that AT1002 upregulates the membrane expression of human major histocompatibility class I and class II molecules (HLA-I; HLA-DR) on monocytes (the numbers in bold represent mean fluorescent intensity values). Interestingly, this activity is exerted at 20 micrograms/ml as well as at a dose 20 fold lower, i.e. 1 microgram/ml. The co-stimulatory molecules CD80 (B7-1) and CD86 (B7.2) are not upregulated on monocytes.

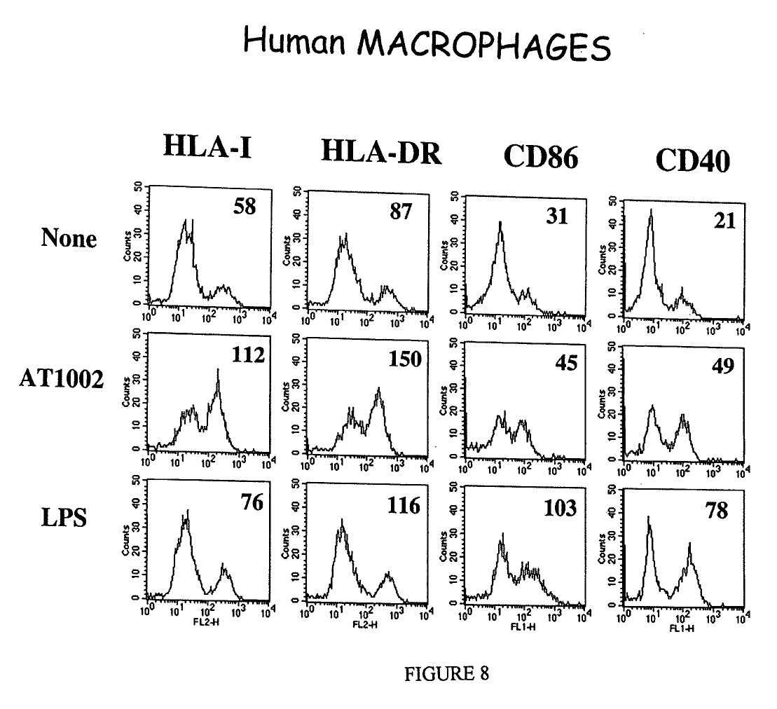

The effects of AT1002 on human macrophages was then analyzed. Human monocytes were purified from peripheral blood of healthy donors and cultured in complete medium for 5 days to allow differentiation into macrophages. Then the stimuli were added to cultures and after 18 hours the cells were harvested, stained with the indicated monoclonal antibodies and analyzed by FACS. The results are shown in FIG. 8. FIG. 8 shows that AT1002 strongly upregulates the membrane expression of HLA-I, HLA-DR and of CD86 (the numbers in bold represent mean fluorescent intensity values). The costimulatory molecule CD80 was also upregulated, although not reported in the figure. In addition, AT1002 upregulates the expression of CD40, a molecule very important for the priming of naïve lymphocytes. The lipopolysaccharide (LPS) was used as a positive control for macrophage activation. In this regard, it should be noted that AT1002 is more efficient than LPS in the upregulation of HLA-I and HLA-DR molecules.

These results demonstrate that AT1002 has immunopotentiating activity. It activates monocytes and macrophages that are antigen presenting cells of the innate immunity important for the stimulation of an antigen-specific immune response. Thus, AT1002 acts as a vaccine adjuvant. Further, the molecules upregulated on monocytes and macrophages are crucial for the stimulation of T lymphocytes. Indeed, HLA I molecules stimulate CD8+ T lymphocytes (cytotoxic cells) that are important to combat intracellular pathogens such as viruses and intracellular bacteria (e.g. Mycobacterium tuberculosis) and against cancer cells; HLA-DR molecules are important for stimulation of the stimulation of CD4+ T lymphocytes that act as a) helper cells that stimulate B lymphocytes to produce antigen-specific antibodies of all classes: IgM, IgG and IgA; and b) as effector cells against infections caused by intracellular and extracellular pathogens. The costimulatory molecules CD80 and CD86 are important for an optimal stimulation of T lymphocytes. The CD40 molecule is also important for the stimulation of antigen-specific T lymphocytes and in particular for the priming of naïve T lymphocytes that express the CD40 ligand molecule.

Without being bound by any theory, it is thought that the mechanism of action of the peptide of the present invention may involve a first step where peptide binds to a receptor located on epithelial cells. The binding modulates tight junctions and allows entry of the co-delivered antigen in the submucosa. Subsequently, the peptide may interact with cells of the immune system to promote/modulate the immune response.

The activity of AT1002 on tight junctions and its effects on antigen presenting cells indicate that AT1002 acts at the same time as a delivery system and as an adjuvant. This is very important for mucosal vaccination, where two important issues are indeed the delivery of the antigen in the submucosa and the stimulation and amplification of an immune response. Generally, two different compounds have to be included in mucosal vaccines to get these two functions, whereas AT1002 has both activities in one molecule.

All patents and publications mentioned in this specification are indicative of the level of those skilled in the art to which the invention pertains. All patents and publications herein are incorporated by reference to the same extent as if each individual publication was specifically and individually indicated to be incorporated by reference in their entirety.

What is claimed is:

1. A method of inducing an immune response in an animal, comprising:
administering to a mucosa of the animal one or more antigens and one or more peptide adjuvants.

2. A method according to claim 1, wherein at least one antigen and at least one peptide adjuvant are administered as a composition.

3. A method according to claim 1, wherein the animal is a mammal.

4. A method according to claim 1, wherein the animal is a human.

5. A method according to claim 1, wherein at least one peptide adjuvant comprises the sequence FCIGRL.

6. A method according to claim 1, wherein at least one peptide adjuvant comprises from about 6 to about 50 amino acids.

7. A method according to claim 1, wherein at least one peptide adjuvant comprises from about 6 to about 25 amino acids.

8. A method according to claim 1, wherein at least one peptide adjuvant comprises from about 6 to about 15 amino acids.

9. A method according to claim 1, wherein at least one antigen is selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, and influenza virus antigens.

10. A method according to claim 2, wherein the composition is in aqueous solution.

11. A method according to claim 2, wherein the composition further comprises one or more pharmaceutically acceptable excipients.

12. A method according to claim 2, wherein at least one peptide adjuvant comprises the sequence FCIGRL and the composition is in aqueous solution and the composition comprises one or more antigens selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, and influenza virus antigens.

13. An immunogenic composition for mucosal administration, comprising:
one or more antigens and one or more peptide adjuvants.

14. A composition according to claim 13, wherein at least one antigen is selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, and influenza virus antigens.

15. A composition according to claim 13, wherein at least one peptide adjuvant comprises the sequence FCIGRL.

16. A composition according to claim 15, wherein the peptide adjuvant comprises from about 6 to about 50 amino acids.

17. A composition according to claim 15, wherein the peptide adjuvant comprises from about 6 to about 25 amino acids.

18. A composition according to claim 15, wherein the peptide adjuvant comprises from about 6 to about 15 amino acids.

19. A composition according to claim 13, wherein the composition is in aqueous solution.

20. A composition according to claim 13, wherein the composition further comprises one or more pharmaceutically acceptable excipients.

21. A composition according to claim 13, wherein at least one peptide adjuvant comprises the sequence FCIGRL and the composition is in aqueous solution and the composition comprises at least one antigen selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, and influenza virus antigens.

22. A vaccine for mucosal administration comprising one or more antigens and one or more peptide adjuvants.

23. A vaccine according to claim 22, wherein at least one antigen is selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, and influenza virus antigens.

24. A vaccine according to claim 22, wherein at least one peptide adjuvant comprises the sequence FCIGRL.

25. A vaccine according to claim 24, wherein the peptide adjuvant comprises from about 6 to about 50 amino acids.

26. A vaccine according to claim 24, wherein the peptide adjuvant comprises from about 6 to about 25 amino acids.

27. A vaccine according to claim 24, wherein the peptide adjuvant comprises from about 6 to about 15 amino acids.

28. A vaccine according to claim 22, wherein the vaccine is in aqueous solution.

29. A vaccine according to claim 28, wherein the vaccine further comprises one or more pharmaceutically acceptable excipients.

30. A vaccine according to claim 22, wherein at least one peptide adjuvant comprises the sequence FCIGRL and the vaccine is in aqueous solution and the vaccine comprises at least one antigen selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, and influenza virus antigens.

31. A method of stimulating antigen presenting cells, comprising:
contacting the antigen presenting cells with an adjuvant peptide.

32. A method according to claim 31, wherein the antigen presenting cells comprise monocytes.

33. A method according to claim 31, wherein the antigen presenting cells comprise macrophages.

34. A method according to claim 31, wherein stimulation results in upregulation of expression of human major histocompatibility class I and class II molecules.

35. A method according to claim 31, wherein stimulation results in upregulation of expression of CD40.

36. A method according to claim 31, wherein the adjuvant peptide comprises the sequence FCIGRL.

37. A method according to claim 31, wherein the adjuvant peptide is present at a concentration of from about 1 μg/ml to about 20 μg/ml.
